(19) United States
(12) Patent Application Publication
(10) Pub. No.: US 2008/0138332 A1
(43) Pub. Date: Jun. 12, 2008

(54) Methods and Compositions for the Treatment and Prevention of Staphylococcus Aureus Infections Through Interference With OPUC Operon Interaction with TRAP

(75) Inventor: Naomi Balaban, Hopkinton, MA (US)

Correspondence Address: JAGTIANI + GUTTAG, 10363-A DEMOCRACY LANE, FAIRFAX, VA, 22030, US

(73) Assignee: Centegen, Inc., Pikesville, MD (US)

(21) Appl. No.: 11/752,630

(22) Filed: May 23, 2007

Related U.S. Application Data
(60) Provisional application No. 60/802,517, filed on May 23, 2006.

(51) Int. Cl. A61K 39/00; A61K 39/395; C07K 16/00

(52) U.S. Cl. 424/133.1; 424/130.1; 424/150.1; 530/387.1; 530/388.1; 530/387.3

(57) ABSTRACT

The bacterial protein OpuCA, an intracellular part of an ABC transporter, has been shown to interact directly with TRAP. The present invention provides methods and compositions directed at interfering with the interaction between OpuCA and TRAP. The resulting inhibition of TRAP advantageously will reduce pathogenesis of all bacteria that utilize this pathway. The present invention further provides methods and compositions directed at interfering with the interaction between TRAP and the extracellular substrate binding protein OpuCC, or the membrane-associated proteins OpuCB and OpuCD, which, like OpuCA, are encoded by the bacterial OpuC operon. Accordingly, the present methods and compositions will be useful in treating diseases caused by such bacteria.

This application claims benefit of the filing date of U.S. patent application Ser. No. 60/802,517, filed May 23, 2006. The entire disclosure of U.S. patent application Ser. No. 60/802,517 is incorporated herein by reference.

### BACKGROUND

1. Technical Field

The invention relates generally to a composition comprising a binding moiety, and more particularly antibodies, that inhibit the interaction between a bacterial protein encoded by the OpuC operon and Target of RNAIII-Activating Peptide (TRAP), as well as vaccines and methods relating to the same to treat or reduce the risk of bacterial infection.

2. Background of the Technology

Staphylococcus aureus is a major human pathogen and is the most common cause of nosocomial pneumonia, surgical site and bloodstream infections, as well as community-acquired infections such as osteomyelitis and septic arthritis, skin infections, endocarditis, and meningitis (1,2). They cause such fatal diseases due to the expression of toxins like Toxic-shock syndrome toxin-1, enterotoxins, hemolysins, and other virulence factors that have been shown to affect the outcome of the infective process (2). The expression of virulence factors is highly regulated and involves cell-cell communication, otherwise known as quorum sensing.

There are two quorum-sensing systems that have so far been described in S. aureus (3) and are referred herein as staphylococcal quorum sensing 1 & 2 (SQS 1 and SQS 2). SQS 1 consists of the autoinducer RNAIII-Activating Protein (RAP) and its target molecule RNAIII-Activating Protein (TRAP) (3,4). At the mid-exponential phase of growth, SQS 1 induces the expression of SQS 2, which is encoded by the accessory gene regulator agr and is composed of agrABCD and hid (RNAIII). AgrD is a pro-peptide that yields an octapeptide pheromone (Autoinducing peptide, AIP) (12) that is processed with the aid of AgrB (13,14). AgrC and AgrA are part of a bacterial two-component system, AgrC being the receptor component that is phosphorylated in an AIP ligand-dependent manner, and AgrA being a regulator (11, 15). RNAIII is a polycistronic transcript, coding for delta hemolysin and acting as a regulatory RNA molecule that upregulates the expression of multiple exotoxins (10).

TRAP is a membrane associated 21 kDa protein that is histidine-phosphorylated, and its phosphorylation is necessary for activation of SQS 2 at the mid-exponential phase of growth. RAP is a 33 kDa protein that activates the agr by inducing the phosphorylation of TRAP (3,4,5,6). An antagonist of RAP, RNAIII-inhibiting peptide (RIP), inhibits the phosphorylation of TRAP and thereby strongly inhibits the downstream production of virulence factors, bacterial adhesion, biofilm formation, and infections in vivo (4,25). Upon disruption of the function of TRAP expression or phosphorylation, the bacteria lose their tendency to adhere and/or ability to form and maintain a biofilm, toxin expression level are reduced and in general, the development and worsening of bacterial indiced diseases is suppressed (7). Functional genomics studies (7,8) indicate that in the absence of TRAP expression or phosphorylation (i.e., a TRAP phenotype), multiple virulence regulatory systems are disrupted, like the global regulatory locus agr (agrABCD and hid [RNAIII]), sarH2, otherwise known as sarU, which is a transcriptional activator of agr (9), and multiple virulence factors. These include alpha, beta, gamma and delta-hemolysin, triacylglycerol lipase precursor, glycerol ester hydrolase, hyaluronate lyase precursor, staphylococcal serine protease (V8 protease), cysteine protease precursor, cysteine protease, staphopain-cysteine proteinase, 1-phosphatidylinositol phosphodiesterase, zinc metalloproteinase aureolysin precursor, holing-like proteins, and capsular polysaccharide synthesis enzymes. Clearly, TRAP belongs to a novel class of signal transducers. Thus, preventing TRAP expression or phosphorylation is a desired result.

The instant invention provides a novel method and composition for prevention and treatment of bacterial infections in general and S. aureus infections in particular.

### SUMMARY

The bacterial protein OpuCA, an intracellular part of an ABC transporter, has been shown to interact directly with TRAP. The present invention provides methods and compositions directed at interfering with the interaction between OpuCA and TRAP. Moreover, the RAP-TRAP interaction noted in the related family cases identified above appears to be facilitated through the trans cell membrane protein OpuC. The resulting inhibition of TRAP advantageously will reduce pathogenesis of any bacteria, like S. aureus, that utilize TRAP in a pathway leading to pathogenesis. As such, the methods and compositions identified herein are useful in treating diseases caused by bacteria that utilize TRAP in a pathway leading to pathogenesis, such as S. aureus.

The present invention further provides methods and compositions effective to inhibit the interaction between TRAP and other proteins encoded by the bacterial OpuC operon, namely the extracellular substrate binding protein OpuCC and the membrane-associated proteins OpuCB and OpuCD.

In a further aspect of the present invention, a vaccine comprises a protein encoded by the OpuC operon or an antigenic fragment thereof The vaccine is administered to an individual, such as a mammal or human, in an amount effective to raise antibodies that are capable of blocking or inhibiting the interaction between TRAP and a protein encoded by the OpuC operon. In one embodiment, the protein encoded by the OpuC operon is OpuCA; however, the protein encoded by the OpuC operon may be any of OpuCA, OpuCB, OpuCC or OpuCD either individually or collectively.

In another aspect of the invention, a pharmaceutical composition is provided that comprises a binding moiety that is capable of binding either a protein encoded by the OpuC operon or TRAP, where the binding reduces the interaction between the protein encoded by the OpuC operon and TRAP. In one embodiment, the binding moiety is an antibody, a fragment thereof, or a compound comprising an epitope-binding region thereof that binds a protein encoded by the OpuC operon or TRAP, where the binding inhibits the interaction between the protein and TRAP. In another embodiment, the binding moiety is a monoclonal antibody, a fragment thereof, or a compound comprising an epitope-binding region of a monoclonal antibody. The antibody, fragment thereof, or compound comprising an epitope-binding region thereof may be humanized. The pharmaceutical composition is administered to an individual, such as a mammal or human, in an amount effective to treat or reduce the risk of a bacterial infection. In one embodiment, the protein encoded by the OpuC operon is OpuCA.

BRIEF DESCRIPTION OF THE DRAWINGS

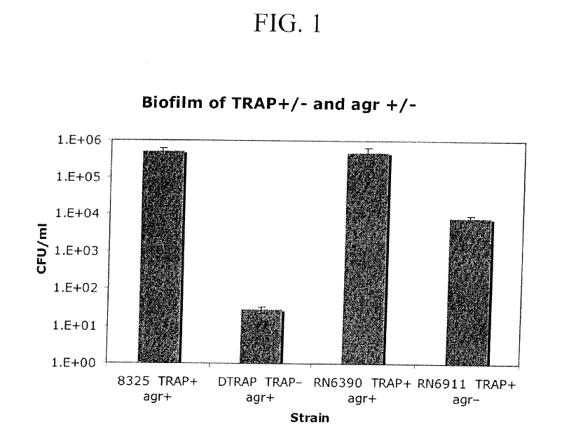

FIG. 1 depicts the results of a comparison of biofilm formation in a rat graft model by mutant strains of S. aureus that display a TRAP− or agr− phenotype.

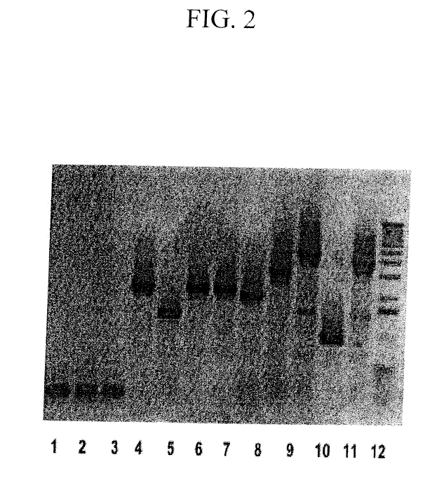

FIG. 2 depicts the generation and validation of the S. aureus 8325-4 genomic library in pTRG. PCR products using pTRG primers for the amplification step are shown. No product was observed from the pTRG only colonies (lanes 1-3), but all 9 pTRG::SAgDNA colonies (lanes 4-12) had PCR products of different sizes ranging from 1-4 kb (Invitrogen 1 kb ladder included on right).

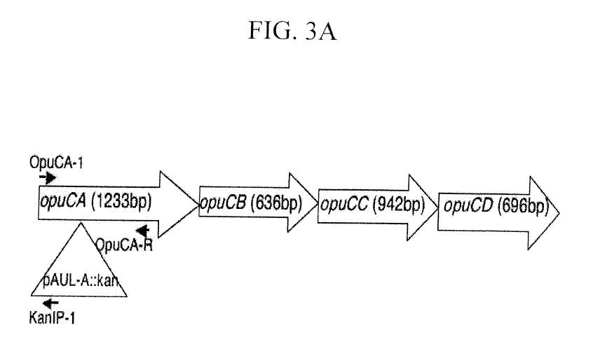

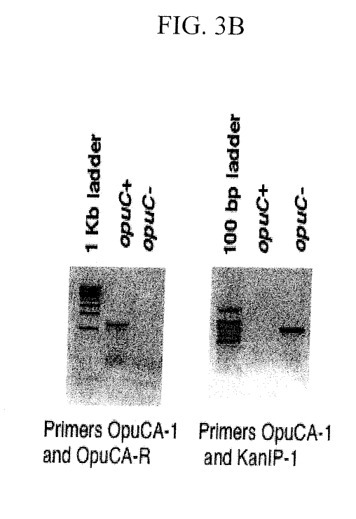

FIG. 3A depicts the opuC locus in S. aureus and the site of pAUL-A integration. The position of primers used is indicated by an arrow.

FIG. 3B depicts PCR analysis indicating the disruption of opuC by insertion of pAUL-A. Left panel: PCR was done using 5′ and 3′ opuCA-1 and OpuCA-R primers. Right panel: PCR was done using opuCA1 and kan1P1 primers. The sequence of the primers is presented in Table 2.

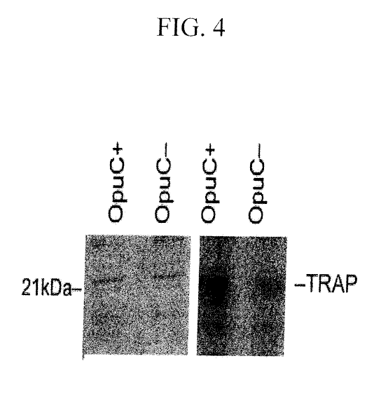

FIG. 4 depicts in vivo phosphorylation. S. aureus OpuC+/− cells were in vivo phosphorylated, total cell homogenate was applied to 15% SDS-PAGE, and the gel was Coomassie-stained (left panel) or autoradiographed (right panel).

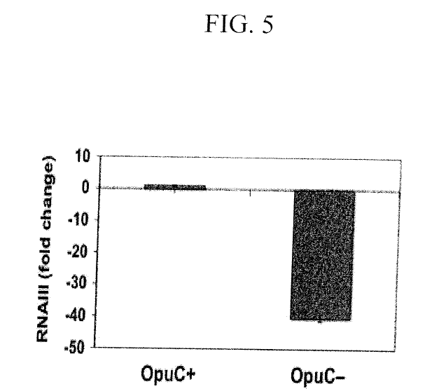

FIG. 5 depicts real time RT PCR on RNA isolated from OpuC+/− cells using primers specific to RNAIII. The sequence of the primers is presented in Table 2.

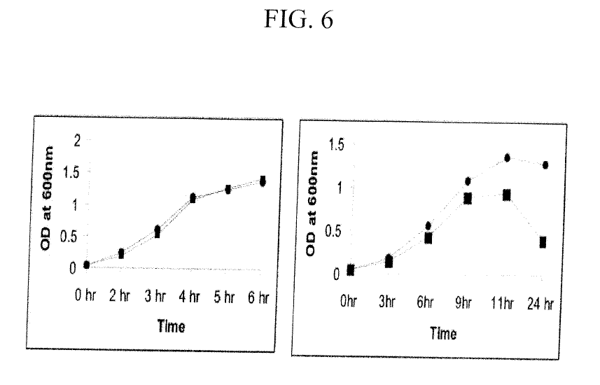

FIG. 6 depicts a growth curve analysis of OpuC+ () and OpuC− (▪) cells grown in LB (left panel) or in LB containing 10% salt (right panel). Cell density was determined spectroscopically at OD 600 nm.

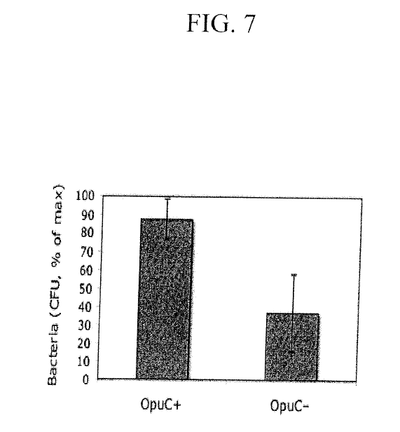

FIG. 7 depicts biofilm formation by OpuC+ and OpuC− cells: Cells were grown in polystyrene 96 well plates for 2 hrs in static conditions in LB at 42° C. Adherent bacteria were stained in gentian violet, solubilized in SDS and OD 595 nm was determined. Results are presented as % of maximum absorbance observed in several experiments.

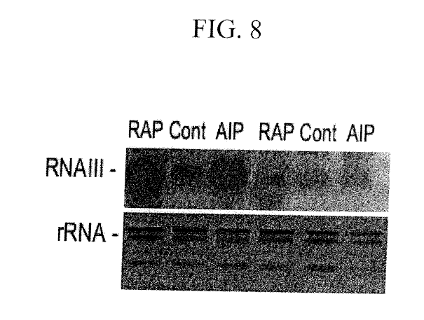

FIG. 8 depicts RNAIII production in OpuC+/˜ cells treated with quorum sensing autoinducers RAP and AIP: Cells were grown in the presence of the autoinducers, RNA isolated, northern blotted, and RNAIII detected using radiolabeled RNAIII-specific DNA. Control: cells grown with <3 kDa spent culture broth of the agr mutant SA RN6911. Bottom panel: Ethidium bromide gel indicating ribosomal RNA loaded.

### DETAILED DESCRIPTION

### TRAP/OpuC Interaction

The present invention provides methods and compositions directed to disrupting the interaction between TRAP and OpuCA as well as TRAP and proteins encoded by the OpuC operon. When the OpuC operon is disrupted, the mutant phenotype displays reduced indicia of pathogenesis, such as reduced TRAP phosphorylation, reduced agr activity, and reduced biofilm formation. Further, OpuCA was shown by a two-hybrid method to interact directly with TRAP. Inhibiting the interaction between TRAP and a protein encoded by the OpuC operon, particularly OpuCA, will treat a presently occurring disease as well as reduce the risk of infection by bacteria in which TRAP plays a role in pathogenesis, such as S. aureus.

There is evidence that TRAP is conserved among all staphylococcal strains and species; therefore, other staphylococcal species should have a quorum sensing mechanism like that described above. In addition, there is evidence of TRAP phosphorylation in S. epidermidis, indicating that there is a similar quorum sensing mechanism in both S. aureus and S. epidermidis. Other infection-causing bacteria appear to have proteins with sequence similarity to TRAP, including Bacillus subtilus, B. anthracis, B. cereus, Listeria innocua, and L. monoctogenes. An inhibitor of the TRAP system thus should interfere with biofilm formation and infections caused by any of these bacterial species.

To search for components of the TRAP system, in this embodiment of the instant invention, TRAP-binding proteins were identified by two hybrid experiments (26, 27), although a number of other methods could be used. Two-hybrid system techniques are well known in the industry. In the present example, S. aureus 8325-4 genomic library was screened twice and clones corresponding to ebh, fmtB and opuCA were identified. Ebh (20) encodes for extracellular matrix binding that is regulated by agr; therefore, based on the self-regulatory nature of the ebh likely acts downstream of TRAP and agr. FmtB (21) encodes a cell wall protein and thus likely acts upstream of TRAP. OpuCA is part of the opuC operon, which was shown in Listeria to encode for an ABC transporter (23); therefore, opuCA likely act upstream of TRAP.

To test which of the candidate proteins interacts with TRAP in a manner important for its activity, OpuC+/− cells, Ebh+/− cells, FmtB+/− cells were tested for TRAP phosphorylation and for hemolytic activity or RNAIII production. SirA+/− cells were used for comparison because SirA encodes for a transporter (22). OpuC− and FmtB− strains were the only ones defective in TRAP phosphorylation, so OpuC and FmtB remained candidates for binding proteins that may have a role in TRAP activity, e.g., regulation of pathogenesis. However, FmtB− cells were hemolytic so this meant that either that the two-hybrid system falsely identified Ebh and FmtB as TRAP-binding proteins, or that, even if these proteins do bind TRAP, they do not disrupt its function. Moreover, the OpuC− cells demonstrated characteristics similar to that of the TRAP−.

OpuCA is encoded by the opuC operon that is highly conserved and was shown in Listeria to encode for a glycine betaine/camitine/choline ABC transporter (23) and is an important osmolyte uptake system contributing to the growth and survival of Listeria both in vitro and in vivo (24). OpuC operon consists of four genes encoding for an ATP binding protein (OpuCA), an extracellular substrate binding protein (OpuCC), and two membrane-associated proteins presumed to form the permease (OpuCB and OpuCD) (23).

In general, ABC (ATP-binding cassette) transporters comprise one of the largest families of structurally related membrane proteins. ABC transporters usually consist of four core domains. Two transmembrane domains form a tunnel and those usually consist of six membrane spanning alpha-helices that contain the substrate binding sites. In addition, ABC transporters possess two highly conserved nucleotide binding domains (NBDs) containing the ATP-binding and -hydrolyzing ‘motor domain’ of the transporter (28).

ABC transporters link ATP hydrolysis to the import or export of various compounds. Bacterial oligopeptide penneases are members of the large family of ATP binding cassette transporters and typically import peptides of 3 to 5 amino acids, apparently independently of sequence. Oligopeptide penneases are needed for bacteria to utilize peptides as nutrient sources and are sometimes involved in signal transduction pathways. In B. subtilis, ABC transporters are also involved in sporulation and competence by importing specific quorum sensing peptide molecules (29) and at least in part by importing specific signaling peptides derived from phr gene products. In staphylococci, there is no documentation of uptake of quorum sensing molecules. The two autoinducers known to date, RAP and AIP, are considered as acting extracellularly, RAP by binding to an unknown receptor to phosphorylate its target protein TRAP and AIP by binding to its receptor AgrC (15).

S. aureus OpuC− showed reduced TRAP phosphorylation, reduced biofilm formation and reduced agr expression, meaning that OpuC acts upstream of TRAP. As OpuC an upstream component to TRAP, the extracellular components of OpuC (OpuCB, OpuCC and OpuCD) may interact with the quorum sensing activators known to regulate TRAP phosphorylation, like RAP and AIP. Indeed, when OpuC+/− cells were grown in the presence of these quorum sensing molecules to test if they can affect TRAP activity, both RAP and AIP activated the production of RNAIII in OpuC+ cells, but did not activate RNAIII in OpuC− cells. In the case of RAP, it is possible that RAP directly interacts with the extracellular components of OpuC.

When OpuC is mutated, the phosphorylation of TRAP is reduced but not abolished. This suggests that there are additional factors regulating TRAP. Such potential partners could be SvrA (30), which is a membrane protein (49 kDa) and has recently been shown to be a component of MATE family efflux pump (31). TRAP− and SvrA− mutants confer essentially identical phenotypes where in both, TRAP is not phosphorylated, agr is not expressed and RNAIII is not produced, and both mutant strains are non-hemolytic (32,30).

Other likely TRAP interacting proteins acting downstream of TRAP include SarH2. SarH2 is a transcriptional factor (9) which activates the agr. Microarray and real time PCR analysis of SarH2 expression in TRAP− cells indicate highly reduced expression as compared to TRAP+ cells (7). SarH2 could thus be a possible downstream protein in RAP/TRAP signaling pathway.

### Antibodies for the Preferred Embodiment of the Instant Invention

The following definitions will be useful for discussing the instant invention:

The term “antibody” refers to an immunoglobulin protein which is capable of binding an antigen. “Antibody” as used herein includes the entire antibody as well as any antibody fragments, e.g., F(ab)′, Fab, Fv, capable of binding the epitope, antigen or antigenic fragment of interest. Preferred antibodies for assays and vaccines of the invention are immunoreactive or immunospecific for, and therefore specifically and selectively bind to, a protein of interest, e.g., an anti-TRAP antibody. Also, as used herein, antibody encompasses all types of antibodies, e.g., polyclonal, monoclonal, humanized, chimeric, and those produced by the phage display methodology. Particularly preferred antibodies of the invention are antibodies which have a relatively high degree of affinity either for TRAP or a protein encoded by the OpuC operon and which inhibit the interaction between these proteins. An “antigenic fragment” of a protein is a portion of such a protein that is capable of binding an antibody.

“Binds specifically” means an antibody binding with high avidity and/or high affinity to an epitope of a specific polypeptide. “Specific binding” is stronger than binding of the same antibody to any other epitope, particularly those which may be present in molecules in association with, or in the same sample, as the specific polypeptide of interest.

A “detectably labeled antibody” is an antibody (or antibody fragment which retains binding specificity) having an attached detectable label. The detectable label is normally attached by chemical conjugation, but where the label is a polypeptide, it could alternatively be attached by genetic engineering techniques. Methods for production of detectably labeled proteins are well known in the art. Detectable labels known in the art include radioisotopes, fluorophores, paramagnetic labels, enzymes (e.g., horseradish peroxidase), or other moieties or compounds which either emit a detectable signal (e.g., radioactivity, fluorescence, color) or emit a detectable signal after exposure of the label to its substrate. Various detectable label/substrate pairs (e.g., horseradish peroxidase/diaminobenzidine, avidin/streptavidin, luciferase/luciferin), methods for labeling antibodies, and methods for using labeled antibodies are well known in the art. See, e.g., Harlow et ah, eds., “Antibodies: A Laboratory Manual,” Cold Spring Harbor Laboratory Press, Cold Spring Harbor, N.Y. (1988).

The present invention provides an antibody that specifically binds and is immunoreactive with TRAP or a protein encoded by the OpuC operon. The antibody may be monoclonal, polyclonal or humanized, and is prepared using methods well known in the art.

Polyclonal antibodies of the present invention may be produced by injecting an animal with TRAP or a protein encoded by the OpuC operon to initiate an immunogenic response. The immunogen may be coupled to a protein carrier such as keyhole limpet hemocyanin (KLH) or bovine serum albumin (BSA). The immunogenicity of the protein may be altered by administering a fragment of the protein that includes less than the entire amino acid sequence of the OpuC protein, the sequence of the protein itself, or the protein and additional sequences. Immunogenicity may also be altered by chemically modifying any of these agents, in addition to the coupling described above, such as by attachment of one or more polyethylene glyucol (PEG) moieties, according to methods known in the art. An adjuvant may also be used. After a suitable amount of time to establish a high-titer of antibodies, the serum or eggs are collected. The presence of antibody in the serum or eggs may be tested by radioimmunoassay (RIA), by enzyme-linked immunosorbent assay (ELISA), or by immunoprecipitation. The immunoglobulins may be isolated by the sequential precipitation methods, by conventional methods of “salting out” the protein fractions from a salt solution, or by chromatographical methods well known to those skilled in the art.

### Candidate Selection to Treat Staphylococcus Infection

Of particular interest in the present invention are agents inhibit TRAP activity by blocking the interaction of TRAP with proteins of the OpuC operon, e.g., any of OpuCA, OpuCB, OpuCC or OpuCD individually or collectively. Such agents are candidates for development of treatments for infection of pathogenic Staphylococcus and other bacteria that utilize the TRAP system. Of particular interest are screening assays for agents that have a low toxicity for human cells and/or high specificity for bacteria, preferably with substantially no or little pressure for selection of strains resistant to the action of the agent, and without substantially affecting normal flora of the host, e.g., as distinguished from wide-spectrum antibiotics.

The term “agent” as used herein describes a protein or pharmaceutical with the capability of altering the interaction of TRAP with a protein encoded by the OpuC operon. A plurality of assay mixtures may be run in parallel with different agent concentrations to detect differential responses to the various concentrations of the agent. Typically, one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, pheromones, purines, pyrimidines, derivatives, structural analogs or combinations thereof

Candidate agents are obtained from a wide variety of sources including libraries of synthetic or natural compounds. For example, numerous means are available for random and directed synthesis of a wide variety of organic compounds and biomolecules, including expression of randomized oligopeptides. Alternatively, libraries of natural compounds in the form of bacterial (e.g., non-pathogenic Staphylococcus), fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

### Screening of Candidate Agents

A wide variety of in vitro assays may be used to screen candidate agents, including labeled in vitro binding assays, e.g., protein-protein binding, electrophoretic mobility shift assays, immunoassays for protein binding, and the like. Purified naturally-occurring or recombinant proteins and/or synthetically produced peptides or fragments of proteins can be used in various screening assays to identify ligands or substrates that bind to, modulate (e.g., increase or inhibit), or mimic the action of the native proteins. The purified proteins may also be used for determination of three-dimensional crystal structure, which can be used for modeling intermolecular interactions, transcriptional regulation, etc.

The screening assay can be a binding assay, wherein one or more of the molecules may be joined to a label that directly or indirectly provides a detectable signal. Various labels include radioisotopes, fluoresces, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures. In general, the particular type of screening assay employed will be amenable to parallel, simultaneous screening of a large number of candidate agents.

One screening assay of particular interest involves detection of TRAP phosphorylation. TRAP phosphorylation screening assays may be performed in, for example, a cell-free assay or in whole cell assays. Phosphorylation screening assays may be performed in a variety of ways. For example, the candidate agent may be combined with detectably labeled phosphate, TRAP, and varying concentrations of RAP to determine if the candidate agent competes with RAP to inhibit TRAP phosphorylation by RAP. Alternatively, the candidate agent may be combined with detectably labeled phosphate, RAP, TRAP, and varying concentrations of RIP to determine if the candidate agent affects RIP activity. Assays, and methods for performing such assays, that can be used in the screening assay of the invention are well known in the art. See, for example, Roychoudhury et al, Proc. Nat'l Acad. Sci. U.S.A. 90: 965-969 (1993), which describes identification of compounds that block the expression of alginate, a virulence factor for the cystic fibrosis pathogen Pseudomonas aeruginosa, and which is incorporated herein by reference with respect to drug screening assays and methods and compositions for performing same.

Screening assays of the present invention also determine the effect of candidate agents on the role of TRAP in RNAIII production and/or virulence factor production. For example, the candidate agent may be contacted with pathogenic Staphylococcus, and the levels of TRAP phosphorylation and/or RNAIII transcription in the presence of the agent compared to TRAP phosphorylation and/or RNAIII transcription levels in the presence of RIP, RAP, and/or a combination of RIP and RAP. Such screening assays can utilize recombinant host cells containing reporter gene systems such as CAT (chloramphenicol acetyltransferase), {3-galactosidase, and the like operably associated with RNAIII or virulence factor genes to facilitate detection of RNAIII or virulence gene transcription or to facilitate detection of RNAIII or virulence factor production. Alternatively, the screening assay can detect RNAIII or virulence factor transcription using hybridization techniques well known in the art, e.g., Northern blot, PCR, etc.

A variety of other reagents may be included in the screening assays described herein. Where the assay is a binding assay, these include reagents like salts, neutral proteins, e.g. albumin, detergents, etc. that are used to facilitate optimal protein-protein binding, protein-DNA binding, and/or reduce non-specific or background interactions. Reagents that improve the efficiency of the assay, such as protease inhibitors, nuclease inhibitors, or anti-microbial agents, may be used. Components are mixed in any order to provide for the requisite binding. Incubations are performed at any suitable temperature, typically between 4° C. and 40° C. Incubation periods are selected for optimum activity, but may also be optimized to facilitate rapid high-throughput screening. Typically between 0.1 and 1 hr will be sufficient.

### Screening of Candidate Agents in an Animal Model

Agents having a desired activity as determined in the assays described above can be further screened for their ability to affect virulence factor production and to affect infection in a non-human animal model. The animal model selected will vary with a number of factors including the particular pathogenic strain against which candidate agents are to be screened, the ultimate host for which the candidate agents are to serve as therapeutics, etc. Animals suitable for use in screening assays include any animal susceptible to infection by the selected pathogenic species. For example, where the bacterial species is S. aureus, the animal model can be a rodent model, preferably a mouse model.

In general, the candidate agent is administered to a non-human animal susceptible to infection, where the animal has been previously infected with the pathogen or receives an infectious does of the pathogen in conjunction with the candidate agent. Preferably, the animal has no detectable antibodies against pathogen proteins. The candidate agent can be administered in any manner desired and/or appropriate for delivery of the agent in order to affect a desired result. For example, the candidate agent can be administered by injection (e.g., by injection intravenously, intramuscularly, subcutaneously, or directly into the tissue in which the desired affect is to be achieved), topically, orally, or by any other desirable means. Normally, this screen will involve a number of animals receiving varying amounts and concentrations of the candidate agent (from no agent to an amount of agent hat approaches an upper limit of the amount that can be delivered successfully to the animal), and may include delivery of the agent in different formulations. The agents can be administered singly or can be combined in combinations of two or more, especially where administration of a combination of agents may result in a synergistic effect.

The effect of agent administration upon the animal model can be monitored by any suitable method, such as assessing the number and size of pathogen-associated lesions, overall health, etc. Where the candidate agent affects bacterial infection in a desirable manner (e.g., by reducing infectious load, facilitating lesion regression, etc.), the candidate agent is identified as an agent suitable for use in treatment of bacterial infection.

### Carrier for Candidate Agents

The compounds having the desired pharmacological activity may be administered in a physiologically acceptable carrier to a host for treatment of pathogenic infection. The therapeutic agents may be administered in a variety of ways including, for example, orally, topically, parenterally e.g. subcutaneously, intraperitoneally, intravascularly, or intrapulmonary. Depending upon the manner of introduction, the compounds may be formulated in a variety of ways. The concentration of therapeutically active compound in the formulation may vary from about 0.1-100 wt. %.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

### Treating Bacterial Infection

The invention provides a method for preventing or treating a human or an animal susceptible to infection by a pathogenic bacteria, e.g., S. aureus in humans, by administering an agent that inhibits the interaction of TRAP with a protein encoded by the OpuC operon. In one embodiment, the host is treated by administration of an agent, such as anti-TRAP antibody, that blocks the interaction. In another embodiment, the agent is co-administered with other TRAP inhibitors and/or co-administered with other inhibitors of bacterial virulence factor production, e.g., RIP. In another embodiment, a TRAP inhibitor, RIP, and a RAP inhibitor, e.g., an anti-RAP antibody, are administered. Such administration of multiple TRAP inhibitory agents may involve co-administration or sequential administration of the active components. The dosage regimen may be adjusted to provide the optimum therapeutic response. For example, several divided doses may be administered daily or the dose may be proportionally reduced as indicated by the therapeutic situation. The active compounds may be administered in any convenient manner, such as by oral, intravenous, intramuscular, subcutaneous, buccal, transdermal, or inhalation routes.

Formulations are administered at a therapeutically effective dosage, e.g., a dosage sufficient to improve the chance of successful prevention or treatment of infection. Human dosage levels for treating infections are known and generally include a daily dose from about 0.1 to 500.0 mg/kg of body weight per day, preferably about 6.0 to 200.0 mg/kg, and most preferably about 12.0 to 100.0 mg/kg. Generally, it is sought to obtain a serum concentration of such a formulation approximating or greater than circulating levels needed to reduce or eliminate any infection in less than 10 days. For administration to a 70 kg person, the dosage range would be about 50 mg to 3.5 g per day, preferably about 100 mg to 2 g per day, and most preferably about 200 mg to 1 g per day. The amount of formulation administered will, of course, be dependent on the subject and the severity of the affliction, the manner and schedule of administration and the judgment of the prescribing physician.

In employing formulation for treatment of infections, any pharmaceutically acceptable mode of administration can be used. The formulations can be administered either alone or in combination with other pharmaceutically acceptable excipients, including solid, semi-solid, liquid or aerosol dosage forms, such as, for example, tablets, capsules, powders, liquids, gels, suspensions, suppositories, aerosols or the like. The formulations can also be administered in sustained or controlled release dosage forms, e.g., employing a slow release bioerodable delivery system, including depot injections, osmotic pumps, pills, transdermal and transcutaneous patches, and the like, for prolonged administration of a predetermined rate, preferably in unit dosage forms suitable for single administration of precise dosages. The compositions will typically include a conventional pharmaceutical carrier or excipient and a formulation of the invention. In addition, these compositions may include other active agents, carriers, adjuvants, etc. Generally, depending on the intended mode of administration, the pharmaceutically acceptable composition will contain about 0.1% to 90%, preferably about 0.5% to 50%, by weight of active compound, the remainder being suitable pharmaceutical excipients, carriers, etc. Methods of preparing such dosage forms are known, or will be apparent, to those skilled in this art. See, e.g., “Remington: The Science and Practice of Pharmacy,” University of the Sciences in Philadelphia, 21^{st} ed., Mack Publishing Co., Easton Pa. (2005).

Parental administration is generally characterized by injection, either subcutaneously, intradermally, intramuscularly, or intravenously, preferably subcutaneously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents, solubility enhancers, and the like, such as for example, sodium acetate, sorbitan monolaurate, triethanolamine oleate, cyclodextrins, and the like.

The percentage of active ingredient contained in such parental compositions is highly dependent on the specific nature thereof, as well as the needs of the subject; however, percentages of active ingredient of 0.01% to 10% in solution are employable, and will be higher if the composition is a solid which will be subsequently diluted to the above percentages. Preferably, the composition will comprise 0.2-2% of the active ingredient in solution. Slow-release or sustained-release systems may be used to deliver a constant dosage. Various matrices, e.g., polymers, hydrophilic gels, and the like, for controlling the sustained release and for progressively diminishing the rate of release of active agents are known in the art.

Formulations of active components may also be administered to the respiratory tract as a nasal or pulmonary inhalation aerosol or solution for a nebulizer, or as a microfine powder for inhalation, alone or in combination with an inert carrier such as lactose, or with other pharmaceutically acceptable excipients. In such a case, the particles of the formulation may advantageously have diameters of less than 50 microns, preferably less than 10 microns. See, e.g., U.S. Pat. No. 5,364,838, which discloses a method of administration for insulin that can be adapted for the administration of formulations of the present invention.

### Vaccination

The invention provides a vaccine for inoculating a human or an animal susceptible to infection by pathogenic bacteria, e.g., S. aureus, by administering TRAP or a protein encoded by the OpuC operon, or an antigenically effective portion of the same, in a pharmaceutically acceptable carrier optionally comprising an adjuvant. Formulations appropriate for eliciting an immune response are well known in the art. In general, the host is exposed to the antigen to perturb the host's immune system and elicit an immune response towards the antigen. An adjuvant can be added with the antigen to increase the immune response to the antigen. The amount of polypeptide administered is an amount sufficient to elicit a protective immune response in the host. Methods for determining such appropriate amounts are routine and well known in the art. For example, an antigenically effective portion can be used to vaccinate an animal that can be used as a model of Staphylococcus infection. The amounts effective in such animal models can be extrapolated to other hosts, e.g., livestock, humans, to provide for an amount effective for vaccination.

### Coated Devices

The invention provides for a device, the surface of which is coated with a composition having an amount of an agent that inhibits the interaction of TRAP with a protein of the OpuC operon in an effective amount to inhibit production of virulence factors by pathogenic bacteria. The coated device may be any device which may be associated with a risk of infection, such as catheters, needles, surgical instruments, e.g., scalpels, sponges, retractors, bandages and bandage materials, e.g., gauze, dressings, artificial joints, heart valves, and tampons. Such devices have a tendency to bring bacteria into contact with the host, or to attract colonizations by bacteria. In such situations, the coated devices may prevent or reduce infection or prevent or reduce the development of serious symptoms associated with exposure to bacterial virulence factors.

### EXAMPLES

### 1. Rat Graft Model

A subcutaneous pocket was made on each side of the median line by a 1.5 cm incision on anaesthetized 250-300 g male Wistar rats (Experiment 1; n=5. Experiment 2: n=15). Aseptically, 1 cm sterile collagen-sealed Dacron grafts (Albograft, Italy) were implanted into the pockets. Pockets were closed by skin clips, and 1 ml saline with or without 2×10 exponentially growing bacterial were inoculated onto the graft surface.

2. In vivo Biofilm Formation by TRAP and agr Mutants

To test biofilm formation by TRAP and agr mutants in vivo, TRAP− and agr− mutants and their parent strains were injected onto the graft and number of bacteria on the graft was counted after 10 days. The results are depicted in FIG. 1. Almost no biofilm was formed by TRAP− mutants (27±5 CFU/ml) compared to the control parent strain S. aureus 8325-4 (5.0±1.1×10⁵ CF/ml). Further, mutated agr (S. aureus RN6911) made reduced biofilm (7.9±1.3×10³) compared to the parent strain S. aureus RN6390 (4.8±1.7×10⁵ CFU/ml). These results confirm that inhibiting TRAP or agr effectively suppresses biofilm formation.

Significantly, biofilm studies in vivo do not correspond with long-term in vitro biofilm studies, which showed enhancement of biofilm production when agr was deleted or repressed. These in vitro studies suggested that QS inhibitors would be inadequate for inhibiting biofilm, perhaps because of continued high expression of bacterial adhesion molecules resulting from the inhibition of agr or TRAP activity. The present studies show clearly that this is not the case in vivo. The present study confirms the efficacy of QS inhibitors to treat biofilm, perhaps because biofilm formation in vivo depends more on expression of exotoxins than on expression of adhesion molecules.

### 3. Other Bacterial Strains and Growth Media.

E. coli XL 1 Blue MRF′ and DH5a were used for cloning purposes and were grown in Luria-Bertani (LB) broth. Protein-protein interactions were studied in E. coli XL 1 Blue MRF′ A(mcrA)183 A(mcrCB-hsdSMR-mrr)173 endAl hisB supE44 thi-1 recAl gyrA96 relAl lac [V laqP HIS3 aadA Kan^{r}], grown at 30° C. in LB broth supplemented with kanamycin (50 ug/ml) and tetracycline (12.5 ug ml), or were grown at 37° C. in minimal medium supplemented with tetracycline (12.5 ug/ml), chloramphenicol (25 fig/ml), streptomycin (12.5 ug/ml) and with 3-amino-1,2,4-triazole (3-AT, 5 mM). S. aureus cells were grown in Tryptic Soy Broth (TSB) or LB containing appropriate antibiotics (Table 1).

### 4. DNA Techniques.

Standard DNA techniques including the use of restriction endonucleases and DNA cloning were performed as described (16). Plasmid DNA from E. coli strains was isolated using the QIAprep Miniprep kit (QIAGEN). Genomic DNA from S. aureus was isolated using the Wizard genomic DNA purification kit (Promega). Recovery of DNA fragments from agarose gels was performed using the QIAquick Gel Extraction kit according to manufacturer's instructions. The DNA was end-repaired (i.e., filled in, kinased or dephosphorylated) using the DNA Terminator Kit (Lucigen).

### 5. Bacterial Two-Hybrid System.

A genomic library screening method based on the BacterioMatch two-hybrid system (Stratagene) was used to identify proteins that interact with TRAP. For this purpose, the TRAP coding sequence was cloned into plasmid pBT (pBT::traP) to serve as bait and S. aureus 8325-4 genomic library (1-4 kb) in pTRG plasmid (pTRG::SA gDNA) was used as target.

5.1 Construction of the Bait Plasmid, pBT::traP.

S. aureus traP gene (GenBank AF202641) was amplified from S. aureus 8325-4genomic DNA (gDNA) by PCR using primers 5′BamHI/TRAP and 3′BglII/TRAP, which contain a Bam HI site or Bgl II site within the 5′ and 3′ primers, respectively (Table 2). Using standard methodology, the amplified traP was digested with Bam HI and Bgl II, then cloned into the bait vector, pBT (Stratagene), which was also digested with Bam HI and Bgl II, to generate pBT′.itraP. A (gly4ser)3 linker was added upstream of the traP gene at Not l/Bam HI site.

5.2 Construction of a S. aureus Genomic DNA Library in pTRG (pTRG::SA gDNA) as Target Vector.

Genomic DNA was isolated from S. aureus strain 8325-4 (a standard laboratory strain) using standard methodology. An aliquot of the gDNA was sonicated and aliquots were removed at times: 1 sec, 3 sec, 8 sec and 12 sec. A small aliquot of gDNA from each time point was run on a 0.8% agarose gel to assess fragment size range. The 8 sec time point had gDNAs ranging from about 1-4 kb and was ligated into the target vector, pTRG which was linearized by digestion with EcoRI and BamHI followed by dephosphorylation.

An aliquot of the final purified DNA preparations was run on a gel to estimate yields (FIG. 2). Ligation reactions (2 with different molar ratios and 1 pTRG only) were set up, transformed into E. coli XL 1 Blue MRF′ Kan^{r} competent cells (Stratagene) and plated onto kanamycin+tetracycline LB plates (30° C.). The pTRG vector only plate had 5 colonies compared to −120 colonies from the pTRG::SA gDNA ligations. Based on the number of colonies, it was estimated that 5,000 colonies were obtained from 1 \x\ of ligation reaction. Therefore, from a 20 \x\ ligation reaction, one should get ˜10⁵ independent colonies, which is ˜5 genome equivalents (assuming 1 in 6 reading frames is correct, with an average insert size of 1 kb).

A 20 ul ligation reaction was set up and transformed as above. The cells were plated onto 150 mm plates and an aliquot was diluted in order to calculate actual library size. The final library size was estimated to be ˜4×10⁵, which represents −20 genome equivalents. The colonies were scrapped off the 150 mm plates and pooled. The cells were divided into 2 aliquots. One aliquot had glycerol added to a final concentration of 15%, then was divided into 1 ml aliquots and stored at −80° C. (master stock). The second aliquot of cells was used to isolate plasmid DNA (pTRG::SA gDNA), to be used for the two-hybrid experiment.

### 5.3 Two Hybrid Protein-Protein Interaction Assays.

For protein-protein interaction assays, transcriptional activation of the HISS gene was used as the initial test for interaction of the bait and target hybrid proteins as follows: Plasmids pBT, pTRG, pBT::traP and pTRG::SA gDNA were co-transformed in a 1:1 w/w ratio in different combinations in the reporter strain E. coli XL 1 Blue MRF′ A(mcrA)183 A(mcrCB-hsdSMR-mrr)173 endAl hisB supE44 thi-1 recAl gyrA96 relAl lac [F′ laqlq HIS3 aadA Kanr]. The combination of pBT/pTRG, pBT::/raP/pTRG, and pBT/pTRG::SA gDNA were considered as negative controls (Table 3). The co-transformants were plated on both non-selective minimal medium and selective minimal medium containing tetracycline (12.5 \igl ml), chloramphenicol (34 jig/ml) and 3-AT (5 mM). The colonies which grew on selective medium were further screened for streptomycin resistance on minimal medium containing tetracycline (12.5 ug/ml), chloramphenicol (34 jig/ml), 3-AT (5 mM) and streptomycin (12.5 ug/ml). To confirm the detected protein-protein interactions, plasmids were isolated from positive clones using Qiagen miniprep kit (Qiagen) and those plasmids were used to transform the reporter E. coli strain with the isolated target plasmid plus bait plasmid on plates containing chloramphenicol, tetracycline, 3-AT and streptomycin. The presence of genomic fragments from these colonies which grew on secondary selection plate was further confirmed by PCR amplification and DNA fragment was identified by DNA sequencing using pTRG-F and pTRG-R primers (Table 2).

6. In vivo Phosphorylation Studies.

S. aureus strains were grown to the early exponential phase (ODeoo 0.2 (equivalent to about 1×10 cells/ml)). The cells from 2 ml cultures were harvested by centrifugation at 4000 g and resuspended in 1 ml of low phosphate buffer (PFB), (3) and 20 uCi of radiolabeled orthophosphate (³²P) (GE Healthcare). Cells were grown for 40 min at 37° C. in and the cells were collected by centrifugation at 12,000 g for 2 min and resuspended in 100 ul of TE buffer Iysostaphin (25 pg/ml), for 10 min at room temperature. Reducing sample buffer (Pierce) was added (without boiling) and sample (total cell lysate) was separated on 15% SDS-PAGE. The gels were autoradiographed and stained in Coomassie to ensure that equal amounts of proteins were loaded on the gel.

### 7. RNA Isolation and Real Time PCR Analysis.

Total RNA was isolated by using modified Qiagen RNeasy™ protect (Qiagen) protocol. S. aureus cells were grown overnight in 3 ml TSB and used as pre-inoculum. These overnight cultures were diluted 1:100 in 5 ml of TSB and the cultures were grown to the post-exponential phase (from ODeoo of 0.03 for 6 hrs) at 37° C. with shaking. The cells were collected by centrifugation and treated with 20 ul of Iysostaphin (2 mg/ml), and 2% SDS containing proteinase K. RNA was extracted with TRIzol method (Sigma-Aldrich) and further purified using Qiagen RNeasy™ protect protocol followed by treatment with DNAse I (Ambion Inc) at 37° C. for 20 min according to manufacturer's instructions. To verify the absence of genomic DNA, PCR was done using these DNAse I treated RNA samples as templates, using 5′ and 3′ specific gene primers. Two micrograms of each RNA samples were used for cDNA synthesis using ImProm-II™ Reverse Transcription System according to manufacturer's instructions (Promega). Random hexamers (Invitrogen) were used to prime the reaction, 1 pl of resulting cDNA reaction was used to set up the real time RT-PCR, using the LightCycler fast start DNA master SYBR Green Kit (Roche), according to manufacturer's instructions. The transcript hid was amplified using RThld primers (Table 2) and the gyrB transcripts that are constitutively expressed were used as an internal control (using RTgyr primers). To monitor the specificity, the PCR products were analyzed by melting curves and agarose gel electrophoresis. The values were normalized with respect to gyrB expression, and data presented in fold change in expression (17) of OpuC−/OpuC+ cells.

8. Construction of S. aureus OpuC− Cells.

The OpuCA gene was insertionally inactivated using the temperature-sensitive suicide vector pAUL-A. This 9.2-kb plasmid carries an erythromycin resistance marker, the pUC19 multicloning site, and a temperature-sensitive replication origin, which enables chromosomal integration events to be selected at a non-permissive temperature (18). Full-length opuCA gene was amplified by PGR using OpuCA-1 and OpuCA-R primers (Table 2) and was cloned into PCR cloning vector pCR2.1 according to manufacturer's instructions (Invitrogen). Plasmids isolated from positive clones were digested with Xbal and Sacl and insert was cloned at Xbal and Sacl sites of pAUL-A similarly digested. Xbal and Sacl fragment contained full length OpuCA along with 40 bp from the vector at the 5′ terminal end. This construct was digested with BamHl which removes 3′ sequence of OpuCA (nearly 750 bp of opuCA). pUTE618 (plasmid containing the omega kanamycin cassette (19) was digested with BamHl, and insert was gel-purified and ligated with the ito/wHI-digested pA\JL-A::opuCA (above). Ligation mix was used to transform into E. coli DH5a and selected on LB agar plates containing kanamycin (100 p.g/ml). The plasmid DNA was isolated from positive clones and used to transform dam− strain of E. coli GM2163 in order to get non-methylated plasmid DNA that was then used to electro transform S. aureus RN4220. Chromosomal integration of pAUL-A::opuCA::kan was selected by repeated plating at 42° C. with selection for erythromycin (30 ug/ml) resistance as described previously (3). The integration of pAUL-A::opuC::Jean into the OpuC locus in RN4220 was confirmed by PCR, and the resulting strain was designated RN4220 AopuC (OpuC−). The primers opuCA-1 and kanIP-1 (Table 2) were used to confirm the single crossing over event that yielded 700-bp DNA fragment.

### 9. Induction of RNAIII Production by RAP and AIP.

RAP and AIP were partially purified as described (3) from culture supernatants of S. aureus RN4220. Briefly, S. aureus 4220 cells were grown from early exponential phases for 6 hrs. Culture broth was collected by centrifugation, lyophilized and resuspended in water to a tenth of the original volume (10×). Material was applied to onto a 3 kDa membrane (Amicon) and fraction less than 3 kDA containing AIP was collected, aliquoted and stored at −70° C. until use. Fraction greater than 3 kDa containing RAP was washed several times with 0.1×PBS using Amicon 10. As a control for spent media, <3 kDa fraction of culture broth of agr null strain S. aureus RN6911 was used. OpuC+/− cells were grown from early exponential phase of growth together with RAP or AIP (final 1× dilution) for 40 min. Cells were collected by centrifugation, RNA purified, northern blotted and RNAIII detected as described (3).

### 10. Biofilm Formation.

S. aureus OpuC+/− cells that were grown overnight in TSB at 37° C. with shaking were diluted 1:100 in TSB, and grown for about 2 hrs to OD₆₀₀ 0.2. 100 ul of these early exponential cells were placed in polystyrene 96 well plates (Falcon) and further grown at 37° C. without shaking for 2 hr. Unattached cells were removed, wells were gently washed with PBS several times, and adherent cells were dried in air. Cells were then fixed with 100% ethanol, stained for 2 min with 0.4% gentian violet in 12% ethanol. Stain was removed, wells were washed several times with PBS, and stained cells were solubilized by the addition of 100 ul 1% SDS. The plate was read at OD 595 nm.

### 11. Osmotolerance.

OpuC+/− cells were grown overnight in LB with shaking at 37° C. They were then diluted 1:100 into LB containing 0, 1%, 5%, 10% and 20% NaCl and grown for several hours with shaking at 37° C. At different time intervals cells were removed and cell density was measured by recording the absorbance at 600 nm.

### 12. Identification of TRAP-binding Proteins.

A genomic library screening method based on the BacterioMatch two-hybrid system (Stratagene) was used to identify proteins that interact with TRAP. For this purpose, the TRAP coding sequence was cloned into plasmid pBT (pBT::traP)to serve as bait and S. aureus 8325-4 genomic library (1-4 kb) in pTRG plasmid (pTRG::gDNA) was used as target vector. The pTRG::SA gDNA bait library was validated by random selection of 9 clones and performing colony PCR (FIG. 2) using pTRG primers. No product was observed from the pTRG-only colonies (FIG. 2 lanes 1-3), but all 9 pTRG::SA gDNA colonies (FIG. 2 lanes 4-12) had PCR products of different sizes ranging from 1-4 kb, indicating that the library is appropriate for further analysis. Transcriptional activation of the HIS3 gene was used as the initial test for interaction of the bait and target hybrid proteins. As shown in Table 3, no colonies grew in control reactions while colonies grew from the pBT::traP+pTRG::SA gDNA reactions. The S. aureus 8325-4 genomic library was screened twice and identified two clones corresponding to ebh and OpuCA (one clone each) in the first screen mdfmtB (three clones) in the second screen. These positive clones were re-transformed and those grew on selective minimal medium, indicating possible protein-protein interactions. Ebh encodes for extracellular matrix binding homologue (clone contained ˜1 kb of 28.6 kb), which is reported to be regulated by agr. FmtB encodes for a cell wall protein (clone contained ˜1 kb of 7.8 kb). OpuCA is part of the opuC operon, which was shown in Listeria to encode for a betaine/carnitine ABC transporter.

To test which of the candidate proteins interacts with TRAP in a manner important for its signaling (TRAP phosphorylation, agr activation and/or hemolysis), S. aureus mutant strains of ebh (20), fmtB (21) and opuC were tested for the above phenotypes. Since there were no reports of opuC mutant of S. aureus, an opuC mutant of S. aureus was generated. Sir A− (22) also was tested because it encodes for an iron siderophore ABC transporter and was used for comparison.

13. Generation of S. aureus OpuC− Mutant.

OpuC operon consists of four genes encoding for an ATP binding protein (OpuCA), an extracellular substrate binding protein (OpuCC), and two membrane-associated proteins presumed to form the pennease (OpuCB and OpuCD) (23). The opuC mutant was generated by insertional mutagenesis of pAUL-A containing a kanamycin cassette into S. aureus 4220 opuCA, resulting in disruption of the whole operon (FIG. 3A). Transformants in which a single crossover recombination event had occurred were further confirmed by PCR using primers OpuCA-1 and OpuCA-R (as shown in FIG. 3AB), which did not yield any fragment in OpuC− as compared to 1.2 kb fragments in case of the parent OpuC+ strain. Further, PCR was performed using primers OpuCA-1 and KanIP-1 (FIG. 3AB), which did not yield any fragment in case of OpuC-f cells but did yield 700 bp fragment in case of OpuC− cells. The sequencing of this 700 bp fragment indicated disruption of the opuC operon. Also, the presence of a transcriptional terminator downstream of omega kanamycin and erythromycin resistance gene on pAUL-A which is oriented with the direction of opuCA transcription as well as the fact that pAUL-A is large (9.2 kb) suggests that the insertion mutation is polar, thereby inactivating the entire opuC operon. The stability of the pAUL-A insertion was confirmed by PCR analysis of cultures grown without erythromycin selection at 30° C. using the above primers. Even after repeated subculturing, no plasmid excision was detected. Of note is that mutant cells grew like the wild type in conventional growth media.

### 14. Phenotypic Screens.

Proteins that interact with TRAP should also have a role in phosphorylating TRAP and/or in activation of agr and pathogenesis. These were tested by in vivo phosphorylation assays, by RNAIII detection, by hemolytic assays and finally biofilm formation.

15. In vivo Phosphorylation Studies.

OpuC+/− cells, SirA+/− cells, Ebh+/− cells and FmtB+/˜ cells were in vivo phosphorylated. As shown in Table 1, OpuC− (FIG. 3) and FmtB− (not shown) cells were the only ones defective in TRAP phosphorylation and thus OpuC and FmtB remained candidates for proteins that interact with TRAP and affect its activity (regulation of pathogenesis).

### 16. Production of RNAIII and Hemolytic Activity.

Once agr is expressed, RNAIII is made, which in turn upregulates the production of hemolysins. Production of hemolysins can be viewed by streaking the cells on blood agar plates and if agr is active, hemolysis can be observed. Some strains, however, like RN4220, are inherently non hemolytic and then the production of RNAIII has to be tested instead.

SirA+/− cells, Ebh+/− cells and FmtB+A− cells were tested for hemolytic activity. As shown in Table 4, all mutant strains were hemolytic, suggesting either that the two-hybrid system falsely identified Ebh and FmtB as TRAP-binding proteins, or that even if these proteins do bind TRAP, they do not disrupt its function. Accordingly, further studies focused only on OpuC.

OpuC− cells were tested for the production of RNAIII by real time PCR. As shown in Table 4 and in FIG. 5, the amount of RNAIII was reduced 40 fold. This suggests that OpuC, which affects TRAP phosphorylation, is involved in pathogenesis.

### 17. Osmotolerance.

Osmolites like glycine betaine and choline chloride are required for salt stress tolerance (24). S. aureus OpuC+/− was therefore tested for salt stress tolerance in LB containing 0, 1, 5, 10 and 20% salt (NaCl). No difference in growth was observed in LB containing additional NaCl of 0, 1 and 5% (FIG. 6A shown for no salt addition) but OpuC− cells were not as tolerant and growth was retarded in 10% NaCl as compared to the wild type (FIG. 6B). Both OpuC+/− cells did not grow in LB containing 20% NaCl. These results suggest that while mutating OpuC did not alter the growth of the cells in LB, it did reduce their ability to grow under stress.

### 18. OpuC is Important for Biofilm Formation.

To test for biofilm formation, OpuC+/− cells were grown on 96 well polystyrene plates overnight and biofilm formation was detected as described (25). As shown in FIG. 7, significantly (p<0.0091) less biofilm was formed by OpuC− mutants, once again suggesting that OpuC plays a role in pathogenesis.

19. Quorum Sensing (QS) Activators Interact with OpuC.

If OpuC is in fact an upstream component to TRAP, the extracellular components of OpuC (OpuCB, OpuCC and OpuCD) may interact with the quorum sensing activators known to regulate TRAP phosphorylation, like RAP and AIP. To test this hypothesis, OpuC+/˜ cells were grown in the presence of these quorum sensing molecules to test if they can affect TRAP activity even in the absence of OpuC. Results shown in FIG. 8 indicate that RAP and AIP activate the production of RNAIII in OpuC+ cells, but do not activate RNAIII in OpuC− cells.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

TABLE 1

| Plasmid or Strain | Characteristics | Reference |
|---|---|---|
| Plasmids | | |
| pAUL-A | Temperature-sensitive E. coli- | (3) |
| | S. aureus shuttle vector | |
| | Ery^{R}, Chl^{R} | |
| pUTE619 | Source for Ω-kan, Kan^{R}, Chl^{R} | (19) |
| pCR2.1 | PCR cloning vector, Kan^{R}, Ap^{R} | Invitogen |
| pUC19 | Cloning vector, Ap^{R} | Lab resource |
| Strains | | |
| E. coli DH5αF′ | Cloning host, Ap | Invitrogen |
| E. coli GM2163 | dcm/dam mutant, Host for | (19) |
| | isolation of unmethylated DNA | |
| | for electroporation into | |
| | S. aureus RN4220 | |
| E. coli TG1 | Cloning vector, Ap^{R} | ZYMO Research |
| E. coli BL-21 DE3 | F⁻ ompT gal [dcm] [lon] hsdSb | Novagen |
| | (r_{B}⁻ m_{B}⁻) | |
| S. aureus RN4220 | Accepts foreign DNA (r⁻ m⁺) | Lab resource |
| S. aureus RN4220 | opuC mutant strain of S. aureus | In this study |
| ΔopuC | RN4220 | |
| S. aureus RN6390 | Prophage-cured wild-type strain | Lab resource |
| S. aureus RN6390 | RN6390 sirAr::Km, Km^{r} | (22) |
| ΔsirA | | |
| S. aureus | RN6390 sirB::Tet; Tet^{r} | (22) |
| RN6390 ΔsirB | | |
| S. aureus RN450 | laboratory strain, Mc^{S} | (21) |
| S. aureus RN450 | Tn551 insertion mutant in fmtB, | (21) |
| ΔfmtB | Em^{r} | |
| S. aureus 8325-4 | Wild-type cured of known | Lab resource |
| | prophages | |
| S. aureus 8325-4, | ebh mutated strain of S. aureus | (20) |
| Δebh | 8325-4 (Em^{r}) | |
| S. aureus 8325-4, | trap mutated strain of S. aureus | (32) |
| ΔtraP | 8325-4 (Kan^{r}) | |

TABLE 2

| | |
|---|---|
| Name | Oligonucleotide Sequence |

| | |
|---|---|
| opuCA-1 | 5′-ACAATTGCGATAATGGTCTTTTT-3′ |
| opuCA-R | 5′-TTACTCGAGTCATGATTTATCATCCC-3′ |
| hid-1 | 5′-GAATTTGTTCACTGTGTCG-3′ |
| hld-2 | 5′-TTTACACCACTCTCCTCAC-3′ |
| KYopuC-Y | 5′-ACGAGACACCATGCAACAAC-3′ |
| RTopuC-K | 5′-CCCACATGTTCTGTTTGCAC-3′ |
| gyr-U | 5′-TTATGGTGCTGGGCAAATACA-3′ |
| gyr-L | 5′-CACCATGTAAACCACCAGATA-3′ |
| KanlP-1 | 5′-GAATTGATCCGGTGGATGAC-3′ |
| pTRG-F | 5′-TCCGTTGTGGGGAAAGTTATC-3′ |
| pTRG-R | 5′-GGGTAGCCAGCAGCATCC-3′ |
| rTRAP-F | 5′-GAATTCCATATGGCTATTAAAAAGTATAA |
| | G-3′ |
| rTRAP-R | 5′-CGCGCGGATCCTTATTTTTTCTTACGTCCAC |
| | G 3′ |
| rOpuCA-F | 5′-ATCTCGAGATGATTATGTTAAGTAT-3′ |
| rOpuCA-R | 5′-ATAGATCTTCATGATTTATCATCTC-3′ |
| 5′BamHI/TRAP | 5′-TTAGGATCCAAGAAACTATATACATCTTATG |
| | GC |
| 3′BGLII/TRAP | 5′-TAAGATCTTAATTAATTAATTATTCTTTTAT |
| | TGGGTATAGATA |
| gly-ser linker A | 5′-ATTGCGGCCGCTGGTGGAGGCTCAGGCGGAG |
| | GTGGCAGCGGCGGTGGCGGATCCTAT |
| gly-ser linker B | 5′-ATAGGATCCGCCACCGCCGCTGCCACCTCCG |
| | CCTGAGCCTCCACCAGCGGCCGCAAT |

TABLE 3
Genetic screen for detection of protein-protein interactions.

| | | 3AT + |
|---|---|---|
| | 3-AT | streptomycin |
| pBT + pTRG | − | − |
| pBT + pTRG::SAgDNA | − | − |
| pTRG + pBT;:/rap | − | − |
| pBT::traP + pTRG::SA gDNA | + | + |

TABLE 4
Phenotypic characterization of the mutants (N/A = not applicable)

| | In vivo | Hemolytic |
|---|---|---|
| Strains | phosphorylation | activity/RNAIII activity |
| S. aureus 8325-4 | Yes | Yes |
| S. aureus 8325-4 AtraP | No | No |
| S. aureus 8325-4 Aebh | Yes | Yes |
| S. aureus RN450 | Yes | Yes |
| S. aureus RN450 AfmtB | Reduced | Yes |
| S. aureus RN6390 | Yes | Yes |
| S. aureus RN6390 AsirA | Yes | Yes |
| S. aureus RN6390 AsirB | Yes | Yes |
| S. aureus RN4220 | Yes | N/A |
| S. aureus RN4220 AopuC | Reduced | Reduced by 40 fold |

### REFERENCES

1. Rubin, R. J., Harrington, C. A., Poon, A., Dietrich, K., Greene, J. A., and Moiduddin, A. (1999) Emerg Infect Dis 5, 9-17.
2. Lowy, F. D. (1998) N Engl J Med 339, 520-532.
3. Balaban, N., Goldkorn, T., Gov, Y., Hirshberg, M., Koyfman, N., Matthews, H. R., Nhan, R. T., Singh, B., and Uziel, O. (2001) J Biol Chem 276, 2658-2667.
4. Balaban, N., Goldkorn, T., Nhan, R. T., Dang, L. B., Scott, S., Ridgley, R. M., Rasooly, A., Wright, S. C, Larrick, J. W., Rasooly, R., and Carlson, J. R. (1998) Science 280, 438-440.
5. Korem, M., Sheoran, A. S., Gov, Y., Tzipori, S., Borovok, I., and Balaban, N. (2003) FEMS Microbiol Lett 223, 167-175.
6. Yang, G., Cheng, H., Liu, C, Xue, Y., Gao, Y., Liu, N., Gao, B., Wang, D., Li, S., Shen, B., and Shao, N. (2003) Peptides 24, 1823-1828.
7. Balaban, N., Stoodley, P., Fux, C. A., Wilson, S., Costerton, J. W., and Dell'Acqua, G. (2005) Clin Orthop Relat Res, 48-54.
8. Korem, M L, Gov, Y., Kiran, M. D., and Balaban, N. (2005) Infect Immun 73, 6220-6228.
9. Manna, A. C, and Cheung, A. L. (2003) Infect Immun 71, 343-353.
10. Novick, R. P., Ross, H. F., Projan, S. J., Kornblum, J., Kreiswirth, B., and Moghazeh, S. (1993) Embo J 12, 3967-3975.
11. Novick, R. P., Projan, S. J., Kornblum, J., Ross, H. F., Ji, G., Kreiswirth, B., Vandenesch, F., and Moghazeh, S. (1995) Mol Gen Genet 248, 446-458.
12. Ji, G., Beavis, R., and Novick, R. P. (1997) Science 276, 2027-2030
13. Qiu, R., Pei, W., Zhang, L., Lin, J., and Ji, G. (2005) J Biol Chem 280, 16695-16704
14. Zhang, L., and Ji, G. (2004) J Bacteriol 186, 6706-6713
15. Lina, G., Jarraud, S., Ji, G., Greenland, T., Pedraza, A., Etienne, J., Novick, R. P., and Vandenesch, F. (1998) Mol Microbiol 28, 655-662
16. J Sambrook, E. F. F., T. Maniatis. (1989) Molecular cloning, Second edition Ed. (Nolan, C, Ed.), Cold spring harbor Laboratory Press, Plainview
17. Livak, K. J., and Schmittgen, T. D. (2001) Methods 25, 402-408
18. Chakraborty, T., Leimeister-Wachter, M., Domann, E., Haiti, M., Goebel, W., Nichterlein, T., and Notermans, S. (1992) J Bacteriol 11 A, 568-574
19. Saile, E., and Koehler, T. M. (2002) J Bacteriol 184, 370-380
20. Clarke, S. R., Harris, L. G., Richards, R. G., and Foster, S. J. (2002) Infect Immun 70, 6680-6687
21. Komatsuzawa, H., Ohta, K., Sugai, M., Fujiwara, T., Glanzmann, P., Berger Bachi, B., and Suginaka, H. (2000) J Antimicrob Chemother 45, 421-431
22. Dale, S. E., Sebulsky, M. T, and Heinrichs, D. E. (2004) J Bacteriol 186, 8356-8362
23. Fraser, K. R., Ilarvie, D., Coote, P. J., and O'Byrne, C. P. (2000) Appl Environ Microbiol 66, 4696-4704
24. Sleator, R. D., Wouters, J., Gahan, C. G., Abee, T., and Hill, C. (2001) Appl Environ Microbiol 67, 2692-2698
25. Balaban, N., Giacometti, A., Cirioni, O., Gov, Y., Ghiselli, R., Mocchegiani, F., Viticchi, C, Del Prete, M. S., Saba, V., Scalise, G., and Dell'Acqua, G. (2003) J Infect Dis 187, 625-630
26. Sun, A. Q., Balasubramaniyan, N., Liu, C. J., Shahid, M L, and Suchy, F. J. (2004) J Biol Chem 279, 16295-16300
27. Slepenkin, A., de la Maza, L. M., and Peterson, E. M. (2005) J Bacteriol 187,473-479
28. Pleban, K., Macchiarulo, A., Costantino, G., Pellicciari, R., Chiba, P., and Ecker, G. F. (2004) Bioorg Med Chem Lett 14, 5823-5826
29. Solomon, J., Su, L., Shyn, S., and Grossman, A. D. (2003) J Bacteriol 185, 6425-6433
30. Garvis, S., Mei, J. M, Ruiz-Albert, J, and Holden, D. W. (2002) Microbiology 148, 3235-3243
31. McAleese, F., Petersen, P., Ruzin, A., Dunman, P. M., Murphy, E., Projan, S. J., and Bradford, P. A. (2005) Antimicrob Agents Chemother 49, 1865-1871
32. Gov, Y., Borovok, I., Korem, M, Singh, V. K., Jayaswal, R. K., Wilkinson, B. J., Rich, S. M., and Balaban, N. (2004) J Biol Chem 279, 14665-14672
33. Frees, D., Qazi, S. N., Hill, P. J., and Ingmer, H. (2003) Mol Microbiol 48, 1565-1578.

What is claimed is:

1. A method of treating or preventing virulent infection in a mammalian host by a staphylococcal bacteria, comprising generating, in said host, a titer of host antibody to a protein expressed by said staphylococcal bacteria infecting or potentially infecting said host, wherein said protein is OpuCA and said titer is effective to suppress virulence in an infection of said host by said staphylococcal bacteria.

2. The method of claim 1, wherein said titer is generated by administering to said host an immunogenic fragment of said OpuCA protein, sufficient to induce said host to express antibodies thereto, wherein said antibodies interfere with interaction between said OpuCA protein and a Target of RNA-II Activating Peptide (TRAP) expressed by said staphylococcal bacteria

3. The method of claim 2, wherein the OpuC protein is comprised of an antigenic fragment that includes less than the entire amino acid sequence of the protein.

4. The method of claim 2, wherein said titer is generated by administering to said host a quantity of anti-OpuCA antibodies effective to achieve said titer in said host.

5. The method of claim 4, wherein said antibodies are administered more than once, so as to sustain said titer over time.

6. A pharmaceutical composition comprising an antibody that binds to a protein encoded by an OpuC operon in a suitable pharmaceutical carrier to treat a condition or a disease in a mammalian host caused by Staphylococcus using TRAP in the Staphylococcus's pathogenesis pathway, wherein the antibody interacts with TRAP to inhibit or retard the Staphylococcus's pathogenesis pathway and the antibody is present in an amount suitable for administration to the host in a dosage range that is therapeutically effective for treating the condition or disease.

7. The pharmaceutical composition of claim 6, wherein the protein encoded by the OpuC operon is OpuCA.

8. The pharmaceutical composition of claim 6, wherein the antibody is monoclonal .

9. The pharmaceutical composition of claim 6, wherein the antibody is humanized.

10. An isolated antibody or antigen binding fragment capable of binding a OpuCA protein encoded by a OpuC operon in a Staphylococcus to treat or prevent a condition or a disease in a mammalian host caused by a pathogenesis pathway involving a TRAP in the Staphylococcus, wherein the isolated antibody or antigen binding fragment binds the OpuCA expressed by the Staphylococcus.

11. The antibody of claim 10, which is a monoclonal antibody.

12. The antibody of claim 10, which is a humanized antibody.

13. The antibody of claim 10, which is an antibody fragment.

14. The antibody fragment of claim 10, which comprises a Fab fragment.

15. The antibody of claim 10, which is an agonist antibody.

16. The antibody of claim 15, which is a humanized antibody.

17. A composition comprising the antibody of claim 10, and a pharmaceutically acceptable carrier.

18. A pharmaceutical composition comprising an immunogenic fragment of said OpuCA protein which induces, when administered to said host, expression of antibodies which bind to said OpuCA protein by said host, in a pharmaceutical carrier.

19. The pharmaceutical composition of claim 18, wherein said fragment is comprised of at least the entire OpuCA protein amino acid sequence.

20. The pharmaceutical composition of claim 17, wherein said fragment is chemically modified to enhance its immunogenicity for said host.
